(19) United States
(12) Patent Application Publication
(10) Pub. No.: US 2002/0156436 A1
(43) Pub. Date: Oct. 24, 2002

(54) Treatment assembly and method of treatment

(76) Inventor: Jeffrey Kerchner, Fleetwood, PA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/837,838

(22) Filed: Apr. 18, 2001

(51) Int. Cl. A61M 35/00

(52) U.S. Cl. 604/290; 604/304

(57) ABSTRACT

The present invention is directed to treatment assemblies and, more specifically, to a treatment assembly having two pockets, one containing a fluid, such as a treatment, and an absorbent material, and the other containing an absorbent material. In one embodiment, the present invention is directed to a treatment assembly, including a first layer and a second layer joined to the first layer to define a first pocket and a second pocket substantially sealed from one another. The assembly further includes a first absorbent material and a fluid in the first pocket and a second absorbent material in the second pocket. The assembly may be used in a method of treatment by removing the first absorbent material from the first pocket and applying the fluid to a treatment area with the first absorbent material. The method may further include removing the second absorbent material from the second pocket and drying the treatment area with the second absorbent material. The present invention may find utility in many applications such a treating skin before taking blood, or cleaning a surface.

### BACKGROUND OF THE INVENTION

1. Field of the Invention

The present invention is directed to treatment assemblies and, more specifically, to a treatment assembly having two pockets, one containing a fluid, such as a treatment, and an absorbent material, and the other containing an absorbent material.

2. Description of the Related Art

In treating a surface, it is common to apply a treatment and then to remove the excess treatment and/or contaminants from the surface. Typically, the treatment is applied with a first absorbent material and removed with a second absorbent material. For example, in treating a patient's skin prior to an injection or other breach, a treatment in the form of an antipathogenic agent or other sterile cleansing agent is typically applied with an absorbent material, such as a swab. In this application, a dry absorbent material, such as a swab, may also be used to blot bodily fluids, such as blood, escaping the breach. As an alternate example, in polishing furniture, a treatment in the form of a wax or other furniture polish is typically applied to the furniture with an absorbent material, such as a cloth. Excess furniture polish and dust may then removed from the furniture using a dry absorbent material, such as a second cloth.

### SUMMARY OF THE INVENTION

In one embodiment, the present invention is directed to a treatment assembly, including a first layer and a second layer joined to the first layer to define a first pocket and a second pocket substantially sealed from one another. The assembly further includes a first absorbent material and a fluid in the first pocket and a second absorbent material in the second pocket.

In another embodiment, the present invention is directed to a treatment assembly, including a first layer and a second layer joined to the first layer to define a first pocket, a second pocket and a third pocket, such that the first pocket, second pocket and third pocket are substantially sealed from one another. The assembly also includes a first sterile, absorbent material and an antipathogenic fluid in the first pocket; a second sterile, absorbent material in the second pocket and a glucose test material in the third pocket.

In another embodiment, the present invention is directed to a treatment assembly, including a first layer and a second layer joined to the first layer to define a first pocket. The assembly also includes a third layer joined to the second layer to define a second pocket substantially sealed from the first pocket. The assembly further includes a first absorbent material and a fluid in the first pocket and a second absorbent material in the second pocket.

In another embodiment, the present invention is directed to a method of treatment. The method includes providing an assembly including a first layer and a second layer joined to the first layer to define a first pocket and a second pocket substantially sealed from one another, a first absorbent material and a fluid in the first pocket and a second absorbent material in the second pocket. The method also includes removing the first absorbent material from the first pocket and applying the fluid to a treatment area with the first absorbent material. The method further includes removing the second absorbent material from the second pocket and drying the treatment area with the second absorbent material.

### BRIEF DESCRIPTION OF THE DRAWINGS

Preferred, non-limiting embodiments of the present invention will be described by way of example with reference to the accompanying drawings, in which:

FIG. 1 is a top, schematic view of an assembly according to one embodiment of the present invention;

FIG. 2 is a top, schematic view of an assembly according to another embodiment of the present invention;

FIG. 3 is a perspective view of an assembly according to another aspect of the present invention;

FIG. 4 is a perspective view of an assembly according to another aspect of the present invention;

FIG. 5 is a top, schematic view of an assembly according to another embodiment of the present invention;

FIG. 6 is a top, schematic view of an assembly according to another embodiment of the present invention;

FIG. 7 is an axial, elevated view of an assembly according to another embodiment of the present invention; and

FIG. 8 is a top, schematic view of the assembly of FIG. 7.

### DETAILED DESCRIPTION OF THE INVENTION

In one embodiment, the present invention is directed to a treatment assembly, including a first layer and a second layer joined to the first layer to define a first pocket and a second pocket substantially sealed from one another. The assembly further includes a first absorbent material and a fluid in the first pocket and a second absorbent material in the second pocket. The assembly may be used in a method of treatment by removing the first absorbent material from the first pocket and applying the fluid to a treatment area with the first absorbent material. The method may her include removing the second absorbent material from the second pocket and drying the treatment area with the second absorbent material. Accordingly, the present invention eliminates the need to apply fluid to an absorbent material before treatment of a surface and the need to have a second absorbent material available to dry the surface, providing a level of convenience not found in the prior art. Furthermore, in embodiments where sterility is required, the present invention may provide ready access to a sterile absorbent material for drying, reducing the chance of infection. In some embodiments, the second absorbent material may be used to blot or dry a bodily fluid, such as blood, escaping a breach in the skin as well as, or instead of, residual fluid from the first pocket.

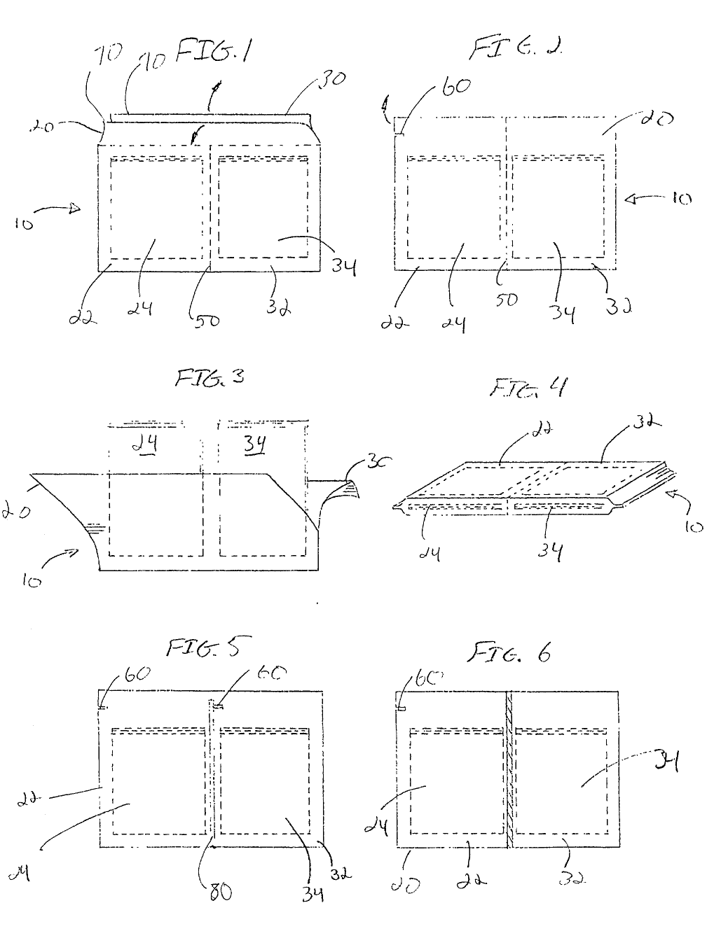

Referring now to the figures, and, in particular, to FIGS. 1 and 2, one embodiment of the present invention will be described by way of example. In this embodiment, a treatment assembly 10, includes a first layer 20 and a second layer 30 joined to first layer 20, defining a first pocket 22 and a second pocket 32. Assembly 10 further includes a first absorbent material 24 and a fluid (not illustrated) in first pocket 22 and a second absorbent material 34 in second pocket 32.

First and second layers 20, 30 may be constructed in any manner and using any materials able to form first and second pockets 22, 32. For example, layers 20, 30 may be formed as any generally planar piece of material, such as a membrane or sheet. In some embodiments, each of layers 20, 30 may include more than one piece of material. For example, first and second layers 20, 30 defining only first pocket 22 may be connected to different first and second layers 20, 30 defining only second pocket 32 and the overall structure still considered to include first and second layers 20, 30. An example of a connection between two sets of layers 20, 30, each defining a pocket 22, 32 is illustrated with cross-hatching in FIG. 6. Such a connection may be performed in any manner that will join the two sets of layers 20, 30, such as the manner in which layers 20, 30 are joined to one another, as will be discussed below.

First and second layers 20, 30 may define the overall shape and size of assembly 10. As assembly 10 may have any overall shape and size, first and second layers 20, 30 may have any overall shape and size. Preferably, first and second layers 20, 30 have matching shapes and sizes, however, in some embodiments, it may be preferred for one of first and second layers 20, 30 to have a different shape, such as, for example, a projecting portion to facilitate the separation of first and second layers 20, 30.

To maximize use of material and ease of production, it is generally preferred that first and second layers 20, 30, and, thus, assembly 10, be generally rectangular. However, the shape of layers 20, 30 may depend, for example, upon the shape of first and second absorbent materials 24, 34 to be contained within pockets 22, 32. For example, the shape of first and second layers 20, 30 may mirror the shape of absorbent materials 24, 34.

Preferably, the cost of construction of layers 20, 30 is minimized, making disposal after a single use more cost effective. For example, the amount of material used to construct layers 20, 30 may be minimized, such as by making layers 20, 30 as thin as possible. In some embodiments, use of a thinner material may also facilitate opening of pockets 22, 32, for example by tearing. The cost and preferred thickness of layers 20, 30 may also depend upon the material used to construct layers 20, 30.

First and second layers 20, 30 may be constructed of any material(s) capable of forming pockets 22, 32 and containing a fluid in first pocket 22. For example, first and second layers 20, 30 may be constructed of a substantially fluid impermeable material compatible with the fluid to be used in first pocket 22. Preferably, first and second layers 20, 30 are constructed of a low cost, durable material to make disposal cost effective and facilitate storage and handling. More preferably, first and second layers 20, 30 are constructed of a material that is substantially impermeable to pathogens, such as bacteria and viruses, and is capable of being sterilized. As used herein, sterile or sterilized means substantially free of microorganisms. The material selected to form first and second layers may also be selected to facilitate opening of pockets 22, 32. For example, the material may be easily torn, or may be strong enough to not break when layers 20, 30 are pulled apart. Materials that may be used to construct first and second layers 20, 30 include metal foils, treated papers, such as metallized paper or plasticized paper, treated or untreated polymeric materials, such as Mylar® polyester films (E. I. duPont de Nemours and Company, Wilmington, Del.) polyethyleneteraphthalate or aluminized Mylar®.

First and second layers 20, 30 may be assembled in any manner that produces pockets 22, 32 having the desired properties for a given application, such as fluid impermeability. Preferably, first and second layers 20, 30 are assembled by joining layers 20, 30 on their sides and at a dividing line 50 between first and second pockets 22, 32. By saying that the layers are joined, it is meant that they are connected to one another in such a way as to allow the pockets 22, 32 to be substantially separate and to allow first pocket 22 to contain a fluid. In some embodiments, it may be desired to join layers 20, 30 only on some sides. For example, where layers 20, 30 are constructed of a single piece of material folded over onto itself to form layers 20, 30, the need for a connection on the side with the fold is avoided.

Layers 20, 30 may be joined by any method that connects layers 20, 30 as required for a given application. For example, layers 20, 30 be mechanically, thermally, frictionally or chemically joined. The method of joining of layers 20, 30 may depend on the material(s) layers 20, 30 are constructed from. For example, where layers 20, 30 are formed of meltable material, they may be heat sealed together. As an alternate example, layers 20, 30 may be adhered to one another by use of an adhesive. Such an adhesive is preferably durable and resistant to the fluid contained within first pocket 22. Other methods of connecting first and second layers 20, 30 will occur to those of skill in the art and are within the scope of the present invention. Depending on the method of connecting first and second layers 20, 30 used, it may be possible to perform the method continuously, forming the individual assemblies as a strip and then separating them.

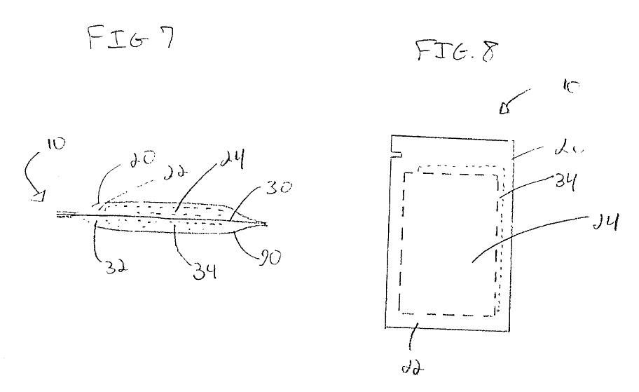

Assembly 10 may be constructed to facilitate opening of pockets 22, 32. Pockets 22, 32 may be opened in any manner that gives useful access to absorbent materials 24, 34. How assembly 10 is typically opened may depend on its construction. For example, where assembly 20 is constructed of a material that may be torn, it may typically be opened by tearing. Where assembly 10 may be opened by tearing, it may include structure to facilitate tearing. For example, as illustrated in FIGS. 2, 5 and 6, assembly 10 may include a pre-cut 60. By pre-cut it is meant a notch, score, or the like, that may serve as a starting place for a tear. Typically, pre-cut 60 is positioned such that one side of pockets 22, 23 may be torn open, for example as illustrated in FIG. 4. Assembly 10 may also include structure to facilitate opening only one pocket 22, 32 at a time. For example, as illustrated in FIG. 5, assembly 10 may include an opening 80 constructed as a notch, or the like, to prevent a tear opening first pocket 22 from extending to second pocket 32 or to facilitate separation of pockets 22, 32 from one another. A perforation or series thereof may also be used to facilitate separation of pockets 22, 32. In this embodiment, a second precut 60 for use in opening second pocket 32 may be included.

Where assembly 10 is constructed such that layers 20, 30 are separable by pulling, pockets 22, 32 may be opened by pulling apart layers 20, 30. Where assembly 10 may be opened by pulling apart layers 20, 30, assembly 10 may include structure to facilitate pulling apart layers 20, 30. For example, as illustrated in FIGS. 1 and 3, assembly 10 may include one or more portions of each layer 20, 30, which may be grasped to pull layers 20, 30 apart, such as pull tabs 70. Typically, pull tabs 70 are positioned along an edge of assembly 10, or at a corner of assembly 10 so that they may be easily grasped.

First and second absorbent materials 24, 34 may be constructed in any manner and of any material(s) that allow them to be contained within first and second pockets 22, 32, respectively, and to absorb a desired amount of liquid. First and second absorbent materials 20, 30 may be constructed in any shape that allows them to be contained within pockets 22, 32 and to be useable for their intended purpose. Typically, first and second absorbent materials 24, 34 will be cut from a larger sheet of material and will be rectangular to maximize the use of the material. However, in some embodiments, absorbent materials 24, 34 may not be shaped to match pockets 22, 32, and, instead, may be compressed, folded, or the like, to fit within pockets 22, 32.

The exact nature of first and second absorbent materials 24, 34 may depend, for example, of the intended use for assembly 10. For example, where assembly 10 is to be used on a delicate surface, such as wooden furniture or sensitive skin, absorbent materials 24, 34 may be soft and non-abrasive. As an alternate example, where some degree of scouring is required, absorbent materials 24, 34 may be abrasive. Similarly, the structural integrity of absorbent materials 24, 34 required for a scouring application may be greater than that for a blotting or disinfecting application. First absorbent material 24 and second absorbent material 34 need not be constructed from the same material. For example, in a cleaning application, first absorbent material 24 may be abrasive while second absorbent material may be soft and non-abrasive. First and second absorbent materials 24, 34 may also be selected based upon the fluid to be used in first pocket 22. For example, absorbent materials 24, 34 may be selected based on their compatibility with the fluid and ability to maintain structural integrity during a desired use. Absorbent materials 24, 34 may also be constructed of material that may be sterilized. Absorbent materials 24, 34 may be designed to remain in contact with a surface to be treated for some period. For example, second absorbent materials 34 may be constructed as an adhesive bandage.

Typically, absorbent materials 24, 34 are constructed from fibrous materials, such as natural or synthetic fiber cloths or swabs, however, other absorbent materials, such as sponge-like materials, may also be used. An example absorbent material for use in assembly 10 is cotton, which may be constructed as a swab or cloth, is relatively inexpensive, soft, absorbent and compatible with most fluids. Various paper products, such as paper toweling may also be used as absorbent material.

The fluid for use in first pocket 22 may be any fluid capable of performing as needed for a particular application. The fluid may include a treatment. For example, where assembly 10 is to be used to clean a surface, the fluid may include a treatment such as a suitable cleaner and where assembly 10 is to be used to provide a skin therapy, the fluid may include a treatment such as a suitable skin therapy. By skin therapy, it is meant any agent beneficial to the skin. As a more specific example, where assembly 10 is for use in treating skin prior to an injection or other breach of the skin, the fluid may include, for example, a sterile cleansing agent, such as sterilized water, or an antipathogenic agent, such as an antibiotic. The antipathogenic agent may be selected based on the pathogens to be eliminated and may be selected not to interfere with any subsequent testing to be done. Common antipathogenic agents for use on human skin include isopropyl alcohol, ethyl alcohol, benzalkonium chloride and povidone iodine. The fluid may also include multiple treatments where the application dictates. For example, if the fluid contains isopropyl alcohol, it may also include a moisturizing agent, such as aloe vera or lanolin, to counter the drying effects of the alcohol. As another example, the fluid may include a makeup remover and a skin toner. The fluid may also include a healing aid. By healing aid it is meant a material that promotes healing. In some embodiments, second pocket 32 may also include some fluid, such as a second treatment agent. For example, second pocket 32 may include a skin therapy such as an antibacterial agent or healing aid, such as Neosporin® antibiotic ointment (Burrows Welcome Co., Research Triangle Park, N.C.).

The present invention is not limited to assemblies including only two pockets 22, 32. For example, the present invention contemplates an assembly including three pockets. In one embodiment of the three pocket assembly, the first two pockets may contain absorbent materials and fluids including treatments, and the third may contain a dry absorbent material. Similarly, the assembly could include four pockets, for example to allow it to be used in a method with two treatment steps and drying after each step. The present invention is also not limited to only pockets containing absorbent material. For example, in some embodiments, assemblies according to the present invention may include other materials for use in a treatment. In one example embodiment, assembly 10 is a diabetic test kit including a third pocket containing a testing medium. As a specific example, the test kit may be a diabetic test kit including a blood glucometer test strip. This embodiment may further include a fourth pocket containing a sterile lancet, or the like, for use in drawing blood for the test.

In another embodiment, illustrated in FIGS. 7 and 8, treatment assembly 10 includes first layer 20 and second layer 30 joined first layer 20 to define first pocket 22. Assembly 10 also includes a third layer 90 joined to second layer 30 to define second pocket 32 substantially sealed from first pocket 22. Assembly 10 further includes first absorbent material 24 and a fluid in first pocket 20 and second absorbent material 34 in the second pocket 32. Additional pockets, for example for use in the applications described immediately above, may be added in this embodiment by adding additional layers to assembly 10. Assembly 10 according to this embodiment may be constructed in the same manner and using the same materials as already described for other embodiments.

The foregoing description is for illustration only and should not be construed as limiting the scope of the invention. The present invention may be embodied in other specific forms without departing from the spirit or essential attributes thereof, and accordingly, reference should be made to the appended claims, rather than to the foregoing specification, as indicating the scope of the invention.

What is claimed is:

1. A treatment assembly, comprising:
a first layer;
a second layer joined to the first layer to define a first pocket and a second pocket, such that the first pocket and the second pocket are substantially sealed from one another;
a first absorbent material and a fluid in the first pocket; and
a second absorbent material in the second pocket.

2. The assembly of claim 1, wherein the first layer and second layer are substantially fluid impermeable.

3. The assembly of claim 1, wherein the first layer is sealed to the second layer on at least one side.

4. The assembly of claim 3, wherein the first layer is heat sealed to the second layer on at least one side.

5. The assembly of claim 1, wherein one of the first layer and the second layer includes a pre-cut.

6. The assembly of claim 1, wherein one of the first layer and the second layer includes a pull tab.

7. The assembly of claim 1, wherein the first layer and the second layer are joined on four sides and along a dividing line to define the first pocket and the second pocket.

8. The assembly of claim 1, wherein the first pocket and its contents are sterile.

9. The assembly of claim 1, wherein the second pocket and its contents are sterile.

10. The assembly of claim 1, wherein the fluid includes antipathogenic material.

11. The assembly of claim 10, wherein the fluid includes isopropyl alcohol.

12. The assembly of claim 1, wherein the fluid includes a skin conditioner.

13. The assembly of claim 12, wherein the fluid includes a moisturizer.

14. The assembly of claim 1, wherein the fluid includes a healing aid.

15. The assembly of claim 1, wherein the second absorbent material includes an adhesive to maintain it in contact with a surface.

16. The assembly of claim 1, wherein the second absorbent material includes a skin treatment.

17. The assembly of claim 16, wherein the second absorbent material includes a healing aid.

18. The assembly of claim 1, wherein the first layer and second layer further define a third pocket containing a testing material.

19. The assembly of claim 18, wherein the testing material includes a glucose test strip.

20. The assembly of claim 1, wherein the first absorbent material is constructed from a different material than the second absorbent material.

21. A treatment assembly, comprising:
a first layer;
a second layer joined to the first layer to define a first pocket, a second pocket and a third pocket, such that the first pocket, second pocket and third pocket are substantially sealed from one another;
a first sterile, absorbent material and an antipathogenic fluid in the first pocket;
a second sterile, absorbent material in the second pocket; and
a glucose test material in the third pocket.

22. The assembly of claim 21, further comprising a fourth pocket defined by the first and second layers and a sterile lancet contained within the fourth pocket.

23. A treatment assembly, comprising:
a first layer;
a second layer joined to the first layer to define a first pocket;
a third layer joined to the second layer to define a second pocket substantially sealed from the first pocket;
a first absorbent material and a fluid in the first pocket; and
a second absorbent material in the second pocket.

24. A treatment assembly, comprising:
a first layer,
a second layer joined to the first layer to define a first pocket and a second pocket, such that the first pocket and the second pocket are substantially sealed from one another;
a first absorbent material and a fluid in the first pocket; and
a glucose testing material in the second pocket.

25. A method of treatment comprising:
providing a treatment assembly including:
a first layer,
a second layer joined to the first layer to define a first pocket and a second pocket substantially sealed from one another,
a first absorbent material and a fluid in the first pocket, and
a second absorbent material in the second pocket;
removing the first absorbent material from the first pocket;
applying the fluid to a treatment area with the first absorbent material;
removing the second absorbent material from the second pocket; and
removing one of the fluid and a second fluid from the treatment area with the second absorbent material.
